(19) United States
(12) Patent Application Publication
(10) Pub. No.: US 2007/0286855 A1
(43) Pub. Date: Dec. 13, 2007

(54) IMPROVING TREATMENTS

(75) Inventors: William J. Sandborn, Rochester, MN (US); William A. Faubion, Oronoco, MN (US); Stephen Ansell, Rochester, MN (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 11/573,165

(22) PCT Filed: Aug. 2, 2005
(86) PCT No.: PCT/US05/27563

§ 371 Date: Jul. 23, 2007

Related U.S. Application Data
(60) Provisional application No. 60/598,369, filed on Aug. 3, 2004.

(51) Int. Cl. A61K 39/395

(52) U.S. Cl. 424/131.1; 424/144.1; 424/178.1

(57) ABSTRACT

Methods and materials for reducing a mammal's ability to produce antibodies against an antigen (e.g., a therapeutic polypeptide) to be administered to the mammal are provided herein.

### BACKGROUND

1. Technical Field

This document relates to methods and materials involved in reducing the production of anti-therapeutic polypeptide antibodies (e.g., anti-immunoglobulin antibodies or anti-idiotypic antibodies) in a mammal.

2. Background Information

Many different therapeutic approaches have been developed to treat illnesses in mammals. For example, the U.S. Food and Drug Administration approved the use of a monoclonal antibody to TNFα (infliximab, Remicade®) to treat Crohn's disease. In many cases, a treatment protocol may achieve less than optimal results for a patient over time because of the patient's immune response to the treatment substance. In fact, infliximab can be immunogenic in humans, limiting its effectiveness.

### SUMMARY

This document relates to methods and materials for reducing a mammal's ability to produce antibodies against a polypeptide (e.g., a therapeutic polypeptide) to be administered to the mammal. Such methods and materials can allow the administered polypeptide to function within the mammal without being neutralized by antibodies produced by the mammal. For example, the methods and materials provided herein can allow the repeated administration of a therapeutic antibody to a mammal (e.g., a human) over time without having the mammal's immune system produce antibodies against the therapeutic antibody such that the beneficial effects of the therapeutic antibody are lost or reduced due to, for example, infusion reactions, hypersensitivity reactions, or loss of efficacy.

In general, one aspect of this document features a method for reducing a mammal's ability to produce antibodies against a therapeutic polypeptide (or non-polypeptide molecule) to be administered to said mammal. The method includes (a) administering an antibody to the mammal, wherein the antibody binds to a B cell surface marker, thereby reducing the number of B cells capable of producing antibodies against the therapeutic polypeptide (or non-polypeptide molecule), and (b) administering the therapeutic polypeptide (or non-polypeptide molecule) to said mammal, wherein the therapeutic polypeptide (or non-polypeptide molecule) is different from the antibody. The mammal can be a human. The therapeutic polypeptide can be a therapeutic antibody. The therapeutic antibody can be a humanized antibody, a fully human antibody, or an antibody fragment. The therapeutic antibody can be a chimeric antibody. The number of B cells capable of producing anti-idiotype antibodies against the therapeutic antibody can be reduced. The antibody can be an anti-CD19, anti-CD20, anti-CD22, anti-CD23, or anti-CD37 antibody. The antibody can be an anti-CD40, anti-B7-1, or anti-B7-2 antibody. The antibody can be administered to the mammal before the therapeutic polypeptide is administered to the mammal. The antibody can be administered concurrently with the therapeutic polypeptide to the mammal. The therapeutic polypeptide can be administered to the mammal before the antibody is administered to the mammal. The therapeutic polypeptide can be administered to the mammal more than two months before the antibody is administered to the mammal. The B cells can be immature B cells. The B cells can be memory B cells.

In another embodiment, this document features a method for reducing a mammal's ability to produce antibodies against an agent to be administered to the mammal. The method includes (a) administering a molecule to the mammal under conditions wherein the number of B cells capable of producing antibodies against the agent is reduced, and (b) administering the agent to the mammal, wherein the agent is different from the molecule. The mammal can be a human. The agent can be a polypeptide. The agent can be an antibody. The agent can be a humanized antibody. The agent can be a fully human antibody, an antibody fragment, or a chimeric antibody. The agent can be infliximab. The molecule can be a monoclonal antibody or a polyclonal antibody. The molecule can be an anti-CD19, an anti-CD20, an anti-CD22, an anti-CD23, an anti-CD37, or an anti-CD40 antibody. The molecule can be an anti-B7-1 antibody or an anti-B7-2 antibody. The molecule can be rituximab. The molecule can be conjugated to a ribosome inactivating protein. The ribosome inactivating protein can be saporin. The molecule can be administered to the mammal before the agent is administered to the mammal. The molecule can be administered concurrently with the agent to the mammal. The agent can be administered to the mammal before the molecule is administered to the mammal. The agent can be administered to the mammal more than two months before the molecule is administered to the mammal. The molecule can be administered to the mammal one time. The molecule can be administered to the mammal multiple times. The B cells can be immature B cells. The B cells can be memory B cells.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 contains graphs plotting the amount (mg/mouse) of mouse anti-goat IgG antibodies detected in serum collected from mice before (pre-immunization) and 24 days after administration of goat IgG. The results are the median and SD of 4 mice.

FIG. 2 contains graphs plotting the number of cells from peripheral blood that are CD19 positive. About 62% of peripheral lymphocytes were B cells as measured by CD19 immunoreactivity (bold line) in control animals versus 28% in mice treated with B cell depleting anti-B220 antibody (filled area).

FIG. 3 is a graph plotting the amount (mg/mouse) of mouse anti-goat IgG antibodies detected in serum collected from mice immunized with 100 μg goat IgG after being either pretreated with rat anti-B220 antibodies or control antibodies. The data represents the median and standard deviation of 4 mice in each group.

FIG. 4 contains graphs plotting the number of cells from peripheral blood that are B220/CD45R positive. About 62% of peripheral lymphocytes were B cells as measured by B220/CD45R immunoreactivity (bold line) in control animals versus 2% in mice treated with B cell depleting saporin-conjugated anti-CD19 antibodies (filled area).

FIG. 5 is a graph plotting the amount (mg/mouse) of mouse anti-goat IgG antibodies detected in serum collected from mice immunized with 100 μg goat IgG after being either pretreated with saporin-conjugated anti-CD19 antibodies or unconjugated anti-CD19 antibodies. Each data point represents an individual mouse.

### DETAILED DESCRIPTION

This document provides methods and materials related to reducing a mammal's ability to produce antibodies against a polypeptide (e.g., a therapeutic polypeptide) or a non-polypeptide molecule to be administered to the mammal. The mammal can be any mammal having the ability to mount an antibody response against foreign antigens. For example, the mammal can be a human. In general, a mammal to receive a polypeptide or non-polypeptide molecule can be treated with a molecule that reduces the mammal's ability to produce antibodies against that polypeptide or non-polypeptide molecule. Molecules having the ability to reduce a mammal's ability to produce antibodies can be, without limitation, antibodies that bind to the mammal's B cells. Examples of such antibodies include, without limitation, anti-CD19, anti-CD20, anti-CD22, anti-CD23, anti-CD37, anti-CD40, anti-B7-1, and anti-B7-2 antibodies.

Antibodies can be any type of antibody such as a human, mouse, rat, goat, sheep, monkey, cow, donkey, or horse antibody. In addition, the antibodies can be produced by cells from any type of organism. For example, antibodies can be obtained from human, mouse, rat, goat, sheep, monkey, cow, donkey, or horse cells. In some cases, the cells (e.g., mammalian, avian, or insect cells) can be cells transfected with nucleic acid encoding an antibody. In one embodiment, the antibodies can be humanized or chimeric (e.g., a human-mouse chimeric) antibodies designed to be administered to a human. In some cases, an antibody (e.g., anti-CD20 antibody) can be conjugated to a ribosome-inactivating protein such as saporin.

Any method can be used to administer antibodies that bind to a mammal's B cells. For example, anti-B cell antibodies such as anti-CD20 antibodies (e.g., rituximab) can be administered to a mammal intravenously, intramuscularly, or intraperitoneally. See, also, U.S. Pat. Nos. 5,595,721 and 6,399,061. Once antibodies designed to bind to a B cell have been administered to a mammal, the mammal's immune system can be monitored to determine whether or not the number of B cells in the mammal has been reduced. For example, a blood sample can be obtained and tested for the presence of B cells. Any method can be used to assess the number of B cells within a sample including standard cell staining or fluorescence-activated cell sorter (FACS) analyses. In some embodiments, the administered antibodies that bind to a mammal's B cells can result in a 5, 10, 15, 20, 25, 30, 40, 50, 60, 70, 80, 90, or more percent reduction in B cells.

Anti-B cell antibodies can be administered once or multiple times to the same mammal. For example, a human can receive daily, weekly, or monthly intravenous injections of rituximab. In some embodiments, a human can receive daily intravenous injections of rituximab for one, two, three, four, five, six, seven, eight, nine, ten, or more weeks.

As provided herein, a mammal to receive a polypeptide (or non-polypeptide molecule) can be treated with a molecule that reduces the mammal's ability to produce antibodies against that polypeptide (or non-polypeptide molecule). This can allow the to be administered polypeptide (or non-polypeptide molecule) to function within the mammal without being partially or completely neutralized by the mammal's antibody response. The polypeptide to be administered can be any type of polypeptide. For example, the polypeptide can be a polypeptide designed to be therapeutic to the mammal. Examples of such polypeptides include, without limitation, polypeptides having enzymatic activity, polypeptides having receptor agonist or antagonist activity (e.g., insulin, growth hormone, GM-CSF, or erythropoietin), and antibodies (e.g., infliximab, MLN-02, natalizumab, CDP870, basiliximab (an anti-IL2 antibody), daclizumab (an anti-IL2 antibody), abciximab, anti-CD44 antibodies, anti-EGFR antibodies, gemtuzumab, trastuzumab, adalimumab, fontalizumab, OKT3 (an anti-CD3 antibody), or visilizumab (an anti-CD3 antibody)). The non-polypeptide molecule to be administered can be any type of non-polypeptide molecule. For example, the non-polypeptide molecule can be a chemical compound designed to be therapeutic to the mammal. Examples of such non-polypeptide molecules include, without limitation, agonists or antagonists of polypeptide activity (e.g., p38 kinase inhibitors, HER-2 phosphorylation inhibitors, MEK inhibitors, or BCR-ABL tyrosine kinase inhibitor STI571).

Any method can be used to administer a polypeptide such as a therapeutic polypeptide to a mammal. For example, anti-TNFα antibodies (e.g., infliximab) can be administered to a mammal intravenously, intramuscularly, or intraperitoneally. See, also, U.S. Pat. Nos. 5,656,272 and 5,698,195.

A mammal to receive a polypeptide or non-polypeptide molecule can be treated with a molecule that reduces the mammal's ability to produce antibodies against that polypeptide or non-polypeptide molecule either before or after the mammal receives the polypeptide or non-polypeptide molecule. For example, anti-B cell antibodies can be administered to a human daily for two weeks before a therapeutic polypeptide such as an anti-TNFα antibody is given to the human. In some embodiments, a human can receive a therapeutic polypeptide for a period of time before being treated with anti-B cell antibodies. In this case, the human can receive additional treatments with the therapeutic polypeptide after receiving the anti-B cell antibodies. In other embodiments, a mammal can be given anti-B cell antibodies concurrently with a therapeutic polypeptide. For example, a human can be treated concurrently with anti-CD20 antibodies and anti-TNFα antibodies.

The invention will be further described in the following examples, which do not limit the scope of the invention described in the claims.

### EXAMPLES

### Example 1

### Depleting Peripheral B Cells

Wild-type, male C57/BL6 mice were purchased and maintained in a pathogen free facility. The wild-type C57/BL6 mice were immunized with 100 μg of goat IgG (Catalog #005-000-002; Jackson ImmunoResearch) mixed 1:1 with Freud's adjuvant by subcutaneous injection at the base of the tail. At 10 days post immunization, wild-type mice were bled (100 μL) by tail vein, and the serum stored for ELISA assay. To measure mouse anti-goat IgG antibodies (MAGA), a sandwich ELISA was developed in the following fashion. ELISA 96 well plates (Nunc Maxisorp) were coated with 5 μg/mL goat IgG (Jackson ImmunoResearch) in 0.1 M carbonate coating buffer pH 9.5 (Pharmingen ELISA protocols). After three washes with PBS/0.05% Tween-20, the wells were then blocked overnight with 10% milk blocking buffer. The study samples were then incubated at varying dilutions for 2 hours, and mouse anti-goat antibodies were detected by HRP-conjugated mouse IgG-Fc fragment antibody (Bethyl Laboratories, Inc. catalog #A90-131P). After standard detection with 1-Step Turbo TMB-ELISA (Catalog #34022; Pierce), the optical density was measured at 450 nm, and the quantity of antibody determined on a Molecular Devices 340PC plate reader using SOFTmax Pro 4.0 software. The lower limit of detection was 50 ng/mL.

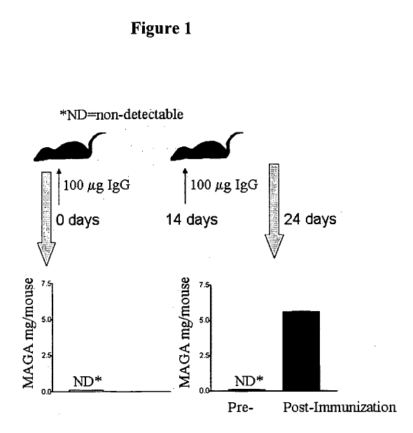

High quantities of MAGA were detected by ELISA. Prior to immunization, wild-type mice had no detectable anti-goat antibody (FIG. 1, left). Mice were immunized at day 0 and boosted with 100 μg of goat IgG at day 14. On day 24, all immunized mice exhibited a vigorous antibody response (FIG. 1, right).

To deplete mice of peripheral B cells, the mice were treated by daily i.p. injection with 100 μg of rat anti-mouse B220/CD45R (Catalog #553083, Pharmingen) for three days, and then twice weekly thereafter. Control mice were treated with control rat IgG2a (Catalog #MAB600, R & D Systems). B cell depletion was assessed by FACS assay of peripheral blood six days after the first injection. Identification of peripheral B cells was made with the fluorescent marker CD19 (Catalog #12-0191, eBioscience). Briefly, peripheral blood mononuclear cells were collected by tail vein bleeding of 100 μL per animal into heparinized collection tubes. Red blood cell lysis was performed using ACK buffer (Biowhittaker, Walkersville, Md.) per manufacturer's protocol. Surface staining and FACS analysis were performed as described previously (Faubion et al., Gastroenterology, 126(7):1759-70 (2004)) using isotype matched control antibody staining as the zero value. All antibodies were purchased from Pharmingen (San Diego, Calif.) or eBioscience (San Diego, Calif.).

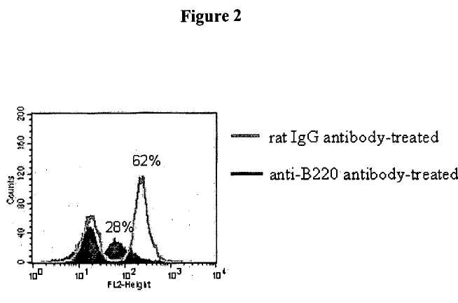

Peripheral B cells were depleted by greater than 50 percent in mice treated with rat anti-mouse B220/CD45R IgG2a antibodies. B220/CD45R is a cell surface receptor expressed on B lymphocytes at all stages from pro-B cells to activated B cells, but not plasma cells. Intraperitoneal injection of 100 μg daily for three days depleted peripheral B cells as measured by FACS on day 6 (FIG. 2). Control animals treated with rat IgG2a have normal levels of B cells (FIG. 2). About 62% of peripheral lymphocytes were B cells as measured by CD19 immunoreactivity in control animals versus 28% in mice treated with B cell depleting anti-B220 antibody (FIG. 2).

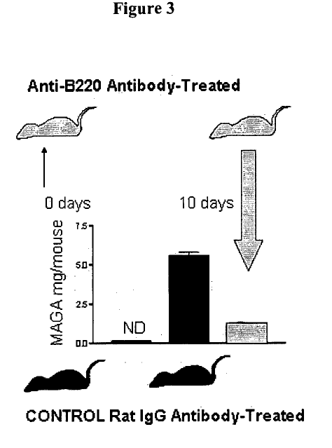

B cell depletion resulted in a markedly impaired murine antibody response to goat IgG. Previously depleted (anti-B220 antibody-treated) or control (rat IgG2a antibody-treated) mice were immunized with 100 μg of goat IgG. Ten days post immunization, the mice were bled, and the serum analyzed for the presence of MAGA antibodies. A severe curtailment in the ability to generate a MAGA response was detected in mice treated with anti-B220 antibodies, but not in control mice (FIG. 3). B cell depletion resulted in an about 80 percent reduction in MAGA production. The data represent the median and standard deviation of 4 mice in each group.

In another experiment, wild-type, male C57/BL6 mice (Harland), that were maintained in a pathogen free facility, were depleted of peripheral B cells. The mice were treated by daily intraperitoneal injection with 100 μg of saporin (ribosome inactivating protein)-conjugated rat anti-mouse CD19 antibody (eBioscience) for three days. Control mice were treated with unconjugated rat anti-mouse CD19 antibody (eBioscience). Saporin conjugation to anti-CD19 antibodies was performed by Advanced Targeting Systems (ATS, San Diego, Calif.). B cell killing was initially assessed in vitro. B cell depletion in vivo was assessed by FACS analysis of peripheral blood six days after the first injection using phycoerythrin-conjugated anti-B220/CD45R antibodies (Pharmingen).

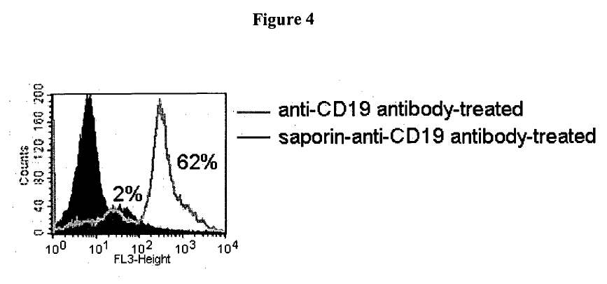

Peripheral B cells were depleted by greater than 95% in mice treated with saporin-conjugated rat anti-mouse CD19 antibody. CD19 is a cell surface receptor expressed on B lymphocytes at all stages from pro-B cells to activated B cells, but not plasma cells. Daily intraperitoneal injection with 100 μg of saporin-conjugated rat anti-mouse CD19 antibody for three days depleted peripheral B cells as measured by FACS on day six (FIG. 4). Control animals treated with unconjugated rat anti-mouse CD19 antibody had normal levels of B cells (FIG. 4). About 62% of peripheral lymphocytes were B cells as measured by B220/CD45R immunoreactivity in control animals versus 2% in mice treated with B cell depleting saporin-conjugated anti-CD19 antibody (FIG. 4).

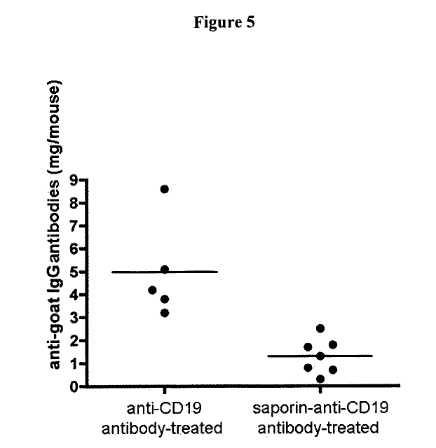

B cell depletion resulted in a markedly impaired murine antibody response to goat IgG. Previously depleted (saporin-conjugated anti-CD19 antibody-treated) or control (unconjugated anti-CD19 antibody-treated) mice were immunized with 100 μg of goat IgG. Ten days post-immunization, the mice were bled, and the serum analyzed for the presence of MAGA. A severe curtailment in the ability to generate a MAGA response was detected in mice treated with saporin-conjugated anti-CD19 antibodies, but not in control mice (FIG. 5).

These results demonstrate that wild-type mice can generate a vigorous antibody response to foreign IgG and that depletion of peripheral B cells is feasible in mammals. These results also demonstrate that pre-treatment with B cell depleting antibodies can severely retard a mammal's ability to generate a vigorous antibody response to foreign antigens.

### Example 2

### Depleting B Cells in Humans to be Treated with Infliximab

Rituximab is a chimeric mouse/human IgG1 monoclonal antibody. It recognizes the CD20 antigen expressed on normal B cells and most malignant B-cell lymphomas. It binds with high affinity to CD20 -positive cells, performs human effector function in vitro, and depletes B cells in vivo. The biological effect is manifested by B-cell depletion in peripheral blood, lymph nodes, and bone marrow. Depletion of memory B cells can allow safe administration of a therapeutic antibody, infliximab, to patients with Crohn's disease who have previously experienced acute or delayed hypersensitivity reactions to infliximab that were treatment limiting but not life threatening, and who have a current indication for treatment with infliximab.

A pilot randomized, double-blind, placebo-controlled trial is performed as follows. The objectives of this study are to demonstrate the safety, tolerability, and pilot evidence of efficacy of pre-treatment with rituximab to induce immunologic tolerance through B cell depletion in patients with Crohn's disease who have previously received treatment with infliximab and have experienced acute or delayed hypersensitivity reactions that were treatment limiting but not life threatening, and who have a current clinical indication for infliximab therapy.

At baseline (week 0), subjects receive the first dose of rituximab 375 mg/m² or placebo followed by a second, third, and fourth dose of rituximab 375 mg/m² or placebo at weeks 1, 2, and 3 respectively. Study medication is administered by i.v. injection using sterile technique.

At weeks 4, 6, and 10, patients receive clinically indicated therapy with a 3 dose induction regimen of infliximab infusions at a dose of 5 mg/kg at weeks 4, 6, and 10. Subjects with previous clinical response to infliximab therapy for Crohn's Disease who have experienced acute or delayed infusion reactions that were treatment limiting but not life threatening, and who have a current indication for infliximab therapy, are enrolled.

A total of 10 subjects having a diagnosis of Crohn's disease with confirmation by endoscopic or radiologic evaluation are enrolled at 1 site and randomized in a 1:1 ratio to pre-treatment with 4 doses of rituximab or placebo and proceed to clinically indicated 3 dose induction of infliximab. Efficacy and safety measurements are performed throughout the study. Blood samples are obtained for the evaluation of infliximab and human anti-chimeric antibody (HACA) concentrations at study entry (week-2), and at weeks 4, 6, 10, and 14. The duration of the study is up to 16 weeks long and includes up to a 2-week screening period and a 14-week treatment period.

Subjects are screened to determine if they meet all of the inclusion criteria and have none of the exclusion criteria. A subject is eligible for study participation if he/she meets the following inclusion criteria: (a) males and females≧18 years of age, (b) female subjects must utilize a highly effective method of birth control throughout the study and for 70 days after study completion, or female subjects must not be of childbearing potential, defined as postmenopausal at least two years, or surgically sterile (bilateral tubal ligation, bilateral oophorectomy, or hysterectomy), (c) a diagnosis of Crohn's disease for greater than 3 months, (d) diagnosis confirmed by endoscopy or radiologic evaluation, (e) previous clinical response to infliximab therapy for Crohn's disease and acute or delayed infusion reactions to infliximab that were treatment limiting but not life threatening (defined as respiratory distress requiring respiratory support or hypotension with >40 mm Hg decrease in systolic BP), (f) subjects who have a current indication for infliximab therapy, (g) subjects must be able and willing to give written informed consent and to comply with the requirements of this study protocol, and (h) adequate cardiac, renal and hepatic function as demonstrated by screening laboratory evaluations, questionnaires, and physical examination results that are within normal limits.

A subject is excluded from the study if he/she meets any of the following criteria: (a) history of cancer or lymphoproliferative disease other than a successfully and completely treated squamous cell or basal cell carcinoma of the skin, (b) subjects with a history of listeria or human immunodeficiency virus (HIV) or a history of previously untreated TB, (c) subjects with ulcerative colitis, (d) subjects with symptomatic obstructive strictures, (e) subjects who have had surgical bowel resections in the past 2 weeks or are planning any resection during the 16 week study, (f) subjects who are currently receiving total parenteral nutrition (TPN), (g) subjects who received any investigational chemical agent in the past 30 days or 5 half-lives prior to screening (whichever is longer), (h) subjects who received any investigational biological agent in 5 half-lives prior to screening, (i) subjects who received antibiotic treatment within 3 weeks prior to screening for all non-Crohn's related infections, (o) female subjects who are pregnant or breast-feeding, (k) history of clinically significant drug or alcohol abuse in the prior year, (l) poorly controlled medical condition, including but not limited to diabetes with documented history of recurrent infections or cerebrovascular accidents (within 3 months), (m) subjects who have been on cyclosporine or tacrolimus within 8 weeks of screening, (n) subjects with detectable serum infliximab concentrations at baseline, (o) subjects with high titer HACA defined as a HACA concentration>8 drawn at the screening visit (week-2), and (p) subjects with the need for live vaccines during the 16 week trial.

Study procedures are performed as summarized in Table 1.

TABLE 1
Schedule of Assessments

| | | | | | | | | Week | Week |
|---|---|---|---|---|---|---|---|---|---|
| | Screening | | Week 1 | Week 2 | Week 3 | Week 4 | Week 6 | 10 | 14 |
| | Day −14 to Day | Baseline | Day 7 | Day 14 | Day 21 | Day 28 | Day 42 | Day 70 | Day 98 |
| | −7 | Day 0 | +3 | +/−3 | +/−3 | +/−3 | +/−3 | +/−3 | +/−3 |
| Inclusion/Exclusion | X | | | | | | | | |
| Criteria | | | | | | | | | |
| Informed Consent | X | | | | | | | | |
| Medical/Surgical Hx | X | | | | | | | | |
| (including | | | | | | | | | |
| Crohn's | | | | | | | | | |
| Medical/Surgical Hx) | | | | | | | | | |
| Previous & | X | | | | | | | | |
| Concomitant | | | | | | | | | |
| Medications | | | | | | | | | |
| Concomitant | | X | X | X | X | X | X | X | X |
| Medication Changes | | | | | | | | | |
| Vital Signs | X | X | X | X | X | X | X | X | X |
| PPD | X | | | | | | | | |
| CXR^{a} | X | | | | | | | | |
| Physical Exam^{b} | X | X | X | X | X | X | X | X | X |
| Pregnancy Test^{c} | X | X | | | | | | | |
| Complete Blood | X | X | X | X | X | X | X | X | X |
| Count | | | | | | | | | |
| Chemistry Group | X | | | | | | | | |
| CRP | | X | X | X | X | X | X | X | X |
| Infliximab Blood | X | | | | | X | X | X | X |
| Sample | | | | | | | | | |
| HACA Blood Samples | X | | | | | X | X | X | X |
| B cell (CD19) | X | X | X | X | X | X | X | X | X |
| concentrations | | | | | | | | | |
| CDAI | | X | X | X | X | X | X | X | X |
| Physician's global | | X | X | X | X | X | X | X | X |
| assessment of disease | | | | | | | | | |
| activity | | | | | | | | | |
| Fistula Assessment | | X | X | X | X | X | X | X | X |
| Adverse Events | | X | X | X | X | X | X | X | X |
| Rituximab | | X | X | X | X | | | | |
| Administration | | | | | | | | | |
| Infliximab | | | | | | X | X | X | |
| Administration | | | | | | | | | |

^{a}CXR - Posteroanterior (PA) and lateral views.
^{b}Height will be measured at Screening only.
^{c}Performed on all women of childbearing potential - serum test at screening and follow-up. Urine neg at Baseline.

All previous and concomitant medications used for Crohn's disease including the highest doses received for each medication are recorded. Medication (prescription) use over the 28 days prior to the screening visit are also recorded. Vital sign determinations of sitting blood pressure, heart rate, respiratory rate and body temperature are obtained at each visit. All subjects undergo purified protein derivative (PPD) placement. All subjects undergo a chest X-ray (CXR), which includes a posteroanterior (PA) and lateral view. Abnormalities detected including granulomatous disease or pleural scarring are reviewed for need for appropriate medical therapy prior to dosing.

A physical examination, including weight, is performed at each visit. Height is measured at screening. The physical examination at the baseline visit serves as the baseline physical examination for the entire study. A count of the number of cutaneous fistulas draining upon gentle compression is performed during the physical exam.

A serum pregnancy test is performed for all female subjects. A urine or blood pregnancy test is performed at baseline. A lactating or pregnant female is not eligible for participation in this study.

Samples are obtained for the laboratory tests listed in Table 2. Blood draws are performed after pain assessments and vital sign determinations during a visit.

TABLE 2
Laboratory tests

| | | | B Cell (CD19) | HACA | Infliximab |
|---|---|---|---|---|---|
| Hematology | Chemistry Group | CRP | Concentration | Concentration | Concentration |
| Hematocrit | Blood Urea Nitrogen | | | | |
| Hemoglobin | (BUN) | | | | |
| Red Blood Cell (RBC) | Creatinine | | | | |
| count | Total bilirubin | | | | |
| White Blood Cell (WBC) | Serum glutamic-pyruvic | | | | |
| count | transaminase | | | | |
| Neutrophils | (SGPT/ALT) | | | | |
| Lymphocytes | Serum glutamic-oxaloacetic | | | | |
| Monocytes | transaminase | | | | |
| Basophils | (SGOT/AST) | | | | |
| Eosinophils | Alkaline phosphatase | | | | |
| Platelet count (estimate | Sodium | | | | |
| not acceptable) | Potassium | | | | |
| | Calcium | | | | |
| | Phosphorus | | | | |
| | Total protein | | | | |
| | Glucose | | | | |
| | Albumin | | | | |
| | Chloride | | | | |

Infliximab and HACA assessments are performed as indicated and are analyzed by an outside laboratory. A Crohn's Disease Activity Index (CDAI) score is calculated from a subject diary and appropriate laboratory values at each visit. A fistula assessment is performed from physical examination at each visit and recorded on a fistula anatomy form. Adverse events are assessed at every study visit starting at baseline. Rituximab and infliximab administrations are given to subjects in the infusion center under the supervision of site medical staff at visits noted on Table 1.

All screening laboratory results are reviewed by the investigator prior to randomization. Subjects who meet the selection criteria proceed to randomization. Subjects are dropped from the study if laboratory or other screening results are unacceptable. At visit 2, patients fulfilling the inclusion/exclusion criteria are randomized according to a computer-generated randomization scheme. Upon notification by the investigator or designee that a patient has qualified for randomization at visit 2, a treatment assignment is made by the un-blinded investigational pharmacist. After a treatment allocation assignment has been made, the patient receives a unique consecutive study number, which is used for identification.

Blood samples for infliximab HACA serum concentrations are obtained at screening (week-2) and at weeks 4, 6, 10, and 14. The time that the blood sample is collected is recorded to the nearest minute in the source document. Blood samples for HACA assay are collected by venipuncture into appropriately labeled 5-mL evacuated serum collection tubes. The assay for HACA antibodies to infliximab is a microplate enzyme-linked immunosorbent assay based on the double-antigen formation in which infliximab is used both during the solid phase to capture antibodies to infliximab and during the biotinylated detection phase with Neutravidin-horseradish peroxidase. The value is reported in micrograms per milliliter on the basis of calculations made with affinity-purified polyclonal rabbit anti-mouse IgG F(ab′). The mean cutoff value in 40 patients who had never received infliximab is 1.69 μg/mL. Antibodies against infliximab (HACA) are reported as negative if the concentration is less than 1.69 μg/mL and the serum infliximab concentration is less than 1.40 μg/mL. Antibodies against infliximab (HACA) are reported as indeterminate when the concentration is less than 1.69 μg/mL but the serum infliximab concentration is 1.40 μg/mL or greater, because infliximab interferes with the assay, these values cannot be conclusively determined. The antibody to infliximab (HACA) test is considered positive when the concentration exceeds 1.69 μg/mL and the serum infliximab concentration is less then 1.40 μg/mL.

The primary pilot efficacy variable is the safe re-administration of infliximab to previously intolerant patients. The secondary efficacy variable measures are: (1) clinical remission (CDAI score<150 points) at each visit; (2) clinical response (a decrease in baseline CDAI score≦100 points) at each visit; (3) improvement in the number of draining fistulas at Week 6 (where improvement is defined as a decrease in the number of draining fistulas≧50% for at least 2 consecutive visits) at each visit; (4) fistula remission at Week 6 (defined as closure of all fistulas that were draining at baseline for at least two consecutive visits) at each visit, (5) CDAI scores at each visit, (6) ability to taper and discontinue steroids according to the tapering schedule after week 10; (7) physician's global assessment of disease severity at each visit; CRP concentrations at each visit.

AEs, laboratory data, physical examinations and vital signs are assessed throughout the study. Reactions to rituximab or infliximab are classified as: (1) acute hypersensitivity reaction (reactions occurring within 2 hours of administration of rituximab/infliximab); (2) delayed hypersensitivity (reactions occurring 1-10 days following administration of adalimumab).

Pre-treatment with rituximab can induce immunologic tolerance through B cell depletion in patients with Crohn's disease who have previously received treatment with infliximab, thereby allowing treatment with infliximab to be effective.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for reducing a mammal's ability to produce antibodies against an agent to be administered to said mammal, said method comprising:
(a) administering a molecule to said mammal under conditions wherein the number of B cells capable of producing antibodies against said agent is reduced, and
(b) administering said agent to said mammal, wherein said agent is different from said molecule.

2. The method of claim 1, wherein said mammal is a human.

3. The method of claim 1, wherein said agent is a polypeptide.

4. The method of claim 1, wherein said agent is an antibody.

5. The method of claim 1, wherein said agent is a humanized antibody.

6. The method of claim 1, wherein said agent is a fully human antibody, an antibody fragment, or a chimeric antibody.

7. The method of claim 1, wherein said agent is infliximab.

8. The method of claim 1, wherein said molecule is a monoclonal antibody.

9. The method of claim 1, wherein said molecule is a polyclonal antibody.

10. The method of claim 1, wherein said molecule is an anti-CD19 antibody, an anti-CD20 antibody, an anti-CD22 antibody, an anti-CD23 antibody, an anti-CD37 antibody, or an anti-CD40 antibody.

11. The method of claim 1, wherein said molecule is an anti-B7-1 antibody or an anti-B7-2 antibody.

12. The method of claim 1, wherein said molecule is rituximab.

13. The method of claim 1, wherein said molecule is conjugated to a ribosome inactivating protein.

14. The method of claim 13, wherein said ribosome inactivating protein is saporin.

15. The method of claim 1, wherein said molecule is administered to said mammal before said agent is administered to said mammal.

16. The method of claim 1, wherein said molecule is administered concurrently with said agent to said mammal.

17. The method of claim 1, wherein said agent is administered to said mammal before said molecule is administered to said mammal.

18. The method of claim 1, wherein said agent is administered to said mammal more than two months before said molecule is administered to said mammal.

19. The method of claim 1, wherein said molecule is administered to said mammal one time.

20. The method of claim 1, wherein said molecule is administered to said mammal multiple times.

21. The method of claim 1, wherein said B cells are immature B cells.

22. The method of claim 1, wherein said B cells are memory B cells.
